(19) United States
(12) Patent Application Publication
(10) Pub. No.: US 2016/0067322 A1
(43) Pub. Date: Mar. 10, 2016

(54) METHOD OF TREATING LUNG CANCER BY VACCINATION WITH MUC-1 LIPOPEPTIDE

(71) Applicant: MERCK PATENT GmbH, Darmstadt (DE)

(72) Inventors: Andreas SCHROEDER, Heidelberg (DE); Christoph HELWIG, Seligenstadt (DE); Anja-Helena LOOS, Oberursel (DE); Armin SCHUELER, Frankfurt am Main (DE); Martin FALK, Roedermak (DE); Charles BUTTS, Edmonton (CA); Frances A. SHEPHERD, Toronoto (CA)

(73) Assignee: MERCK PATENT GmbH, Darmstadt (DE)

(21) Appl. No.: 14/891,557

(22) PCT Filed: Apr. 14, 2014
(86) PCT No.: PCT/EP2014/000992

§ 371 Date: Nov. 16, 2015

(30) Foreign Application Priority Data
May 14, 2013 (EP) 13002525.7

(51) Int. Cl. A61K 39/00; A61N 5/10; A61K 45/06; A61K 39/39; A61K 9/127; A61K 33/24

(52) U.S. Cl. CPC A61K 39/0011; A61K 39/0012; A61K 9/127; A61K 33/24; A61K 45/06; A61K 39/39; A61N 5/10; A61K 2039/55572

(57) ABSTRACT

The invention is directed to the treatment of lung cancer, preferably non-small cell lung cancer (NSCLC) by means of a combination therapy comprising concurrent chemo-radiotherapy followed by vaccination with a muc-1 lipopetide. The therapy elicits prolonged survival rates compared to a respective therapy including sequential chemo-radiotherapy.

### FIELD OF THE INVENTION:

The invention is directed to the treatment of lung cancer, preferably non-small cell lung cancer (NSCLC) by means of a combination therapy comprising concurrent chemo-radiotherapy followed by vaccination with a muc-1 lipopeptide. The therapy elicits prolonged survival rates compared to a respective therapy applying sequential chemo-radiotherapy.

### BACKGROUND OF THE INVENTION

Lung cancer is the leading cause of cancer death in men, with an overall 5-year survival rate of approximately 10 to 15%. The limited efficacy and the toxicity associated with chemotherapy for non-small cell lung cancer (NSCLC) has created a need for safer and more efficacious treatment options. With the identification of tumor-associated antibodies and antigens (TAA) in patients with lung cancer, immunotherapy has emerged as an attractive alternative.

Mucin 1 (MUC1) is one such TAA that is an epithelial glycoprotein overexpressed in NSCLC. T-cells specific for antigenic epitopes of MUC1 that bind to HLA class I molecules have been identified and isolated from the blood and bone marrow of cancer patients (Bared et al., Proc Natl Acad Sci USA. 1989;86:7159-7163; Choi et al. Blood. 2005;105:2132-2134).

The immune-dominant peptides from the variable number of tandem repeat region (VNTR) are recognized by the cytotoxic T-lymphocytes (CTL), making MUC1 an attractive target for therapeutic intervention. The repeating peptide unit is built by 20 amino acid: STAPPAHGVTSAPDTRPAPG.

A number of studies have shown that MUC1 may facilitate epithelial carcinogenesis. High MUC1 expression in tumors has been correlated with increased invasiveness, migration, and angiogenesis in ovarian and lung cancers. Depolarized expression of MUC1 has been related to poor prognosis in early stage NSCLC (Gao et al. Int J Oncol. 2009;35:337-345). Recent findings have indicated that NSCLC cells are dependent on the MUC1-C terminal cytoplasmic domain for both activation of the phosphatidylinositol 3-kinase (PI3K)-Akt pathway and for survival (Raina et al. Mol Cancer Ther. 2011;10:806-816).

A number of studies are focused on devising techniques to effectively present MUC1 as an immunogenic agent to stimulate a strong and highly specific immune response against target cells over-expressing MUC1. L-BLP25 is one such innovative liposomal antigen-specific cancer immunotherapy currently under development that contains 25 amino acids from the immunogenic tandem-repeat region of MUC1 (Mehta et al.,Clin Cancer Res. 2012;18:2861-2871): STAPPAHGVTSAPDTRPAPGSTAPP (SEQ ID No. 1).

L-BLP25 is an active immunotherapeutic agent designed to induce a cellular immune response by targeting T-cell epitopes from the VNTR region of the MUC1 antigen associated with HLA class I molecules. Although NSCLC is historically regarded as a non-immunogenic cancer, L-BLP25 in phase II clinical trials has shown survival advantages with a remarkably low toxicity profile (WO 2005/112546; Butts et al.,J Cancer Res Clin Oncol. 2011;137:1337-1342). In these trials a single, low, intravenous dose (300 mg/m² to a maximum of 600 mg) of cyclophosphamide (CPA) is administered three days prior to immunotherapy. This procedure is thought to enhance delayed-type hypersensitivity humoral and cellular immune responses by reducing T-suppressor function. Although CPA lacks any significant activity in NSCLC, and the dose used in this setting is below that used in cytotoxic chemotherapy, it is currently believed that the observed antitumor effects following L-BLP25 therapy can be attributed to the immunomodulatory effects of CPA.

Nonetheless, there is a continuous need to improve the efficacy of BLP25 or related muc-1 lipopeptides and develop alternative treatment regimen which cause prolonged survival time of a lung cancer patient.

### SUMMARY OF THE INVENTION

It has been found by the inventors that in a comprehensive statistically based clinical trial vaccination with the known muc-1 lipopeptides, preferably BLP25, is effective in combination with chemo-radiotherapy if applied to a cohort of lung tumor patients, preferably, patients suffering from non-small cell lung cance (NSCLC), and most preferably patients suffering from unresectable stage III NSCLC. However, the chemo-radiotherapy is much more effective if the chemo-radiotherapy approach is started before vaccination, and chemotherapy and radiotherapy are carried out concurrently/simultaneously or timely overlapping by at least 30 -50% calculated of the chemotherapy duration. In contrast, and surprisingly, the efficacy of vaccination with said muc-1 lipopeptides as specified in this invention is strongly reduced and if any only slightly increased versus the same treatment with a placebo if the radiation therapy is started after completion of the chemotherapy or is timely overlapping with chemotherapy by less than 10% of the duration of the treatment with chemotherapeutic agents. The best results can be obtained according to the invention if—within a certain range—a specific administration and treatment regimen is applied as described in the specification and the claims. Thus, the statistic overall survival time (OS) of a patient group can be extended by at least 15%, preferably at least 30% and most preferably between 25 and 50%. In parallel, and in addition the time of disease progression (TTP) is also prolonged between 15% and 50% by average.

Moreover, surprisingly, the vaccination with said muc-1 lipopetide formulations of the invention after completion of a sequential chemo-radiotherapy provokes no significant effect with regard to OS/TTP versus the administration of a placebo, whereas the same setting in a concurrent chemo-radiotherapy causes a prolongation of 25 -60% compared to the respective placebo administration.

Therefore, the invention is related to a liposomal formulation comprising a lipopeptide based on the muc-1 core repeating unit selected from the group consisting of the amino acid sequences:

(SEQ ID No. I)
STAPPAHGVTSAPDTRPAPGSTAPP
or
(SEQ ID No. II)
STAPPAHGVTSAPDTRPAPGSTAPP-K-palmitoyl-(G)

for use in the treatment of lung cancer in combination with chemo-radiotherapy, wherein the treatment comprises concurrent chemo-radiotherapy followed by vaccination with the liposomal formulation, wherein the treatment causes an overall-survival (OS) and/or a time-to-progress (TTP), which is prolonged by at least 15%, preferably at least 30%, and most preferably between 25 and 50% compared to an analogous sequential chemo-radiotherapy treatment.

The invention is also related to a respective use of said liposomal formulation, wherein the treatment causes an OS and/or a TTP, which is prolonged between 15 -50% compared to the analogous sequential chemo-radiotherapy, and in addition, 25 -60% compared to an analogous concurrent chemo-radiotherapy treatment, wherein a placebo is applied instead of the liposome vaccine formulation.

The invention relates further to said use of said liposomal formulation, wherein the chemotherapy is carried out by administering chemotherapeutic agents including at least one platinum based chemotherapeutic compound, preferably cisplatin or carboplatin. In addition further chemotherapeutic agents can be applied and may be helpful.

The invention is further related to said use of said liposomal formulation, wherein an adjuvant is applied together with the liposomal vaccine formulation. In a preferred embodiment, the adjuvant is part of the liposome that contains the muc-1 lipopetide or integrated into the liposome.

The liposomal formulation of the invention comprises preferably an adjuvant, which is selected from the group consisting of MPL(3-Odesacyl-4′-monophosphoryl lipid), Lipid A, or low-toxic variants of LPS. MPL is most preferred.

The invention is specifically directed to a liposomal formulation, wherein the muc-1 lipopetide is based on SEQ ID NO. 2. The respective liposomal MPL-lipopetide formulation is designated as L-BLP25.

The liposomal formulation according to the invention is effective in vivo in patients suffering from lung cancer, preferably non-small cell lung cancer (NSCLC), and most preferably unresectable stage III NSCLC. Nonetheless, it cannot be excluded that the treatment as provided can be successfully used in the treatment of cancers different from lung cancer, such as breast or prostate cancer and the like.

According to the invention, the liposomal formulation can be administered in combination with at least a further pharmaceutically effective anti-cancer agent.

Furthermore, the invention is related to a method of treating a patient suffering from lung cancer, preferably NSCLC, more preferably unresectable stage III NSCLC, comprising the following steps:

(i) applying chemo-radiotherapy to said patient, wherein said chemotherapy, preferably platinum-based chemotherapy, preferably cisplatin or carboplatin, and said radiotherapy is carried out concurrently or at least timely overlapping, preferably by at least 10%-100%, preferably 20-100%, most preferably 70-100% related to the duration of the chemotherapy, and

(ii) vaccinating said patient after completion of said chemo-radiotherapy every 5^{th}-9^{th} day, preferably every 7^{th} day for at least 4-8 administrations, and every 35^{th}-49^{th} day, preferably every 42^{nd} day for the following period, with a liposomal formulation comprising a lipopeptide based on the muc-1 core repeating unit selected from the group consisting of the amino acid sequences:

(SEQ ID No. I)
STAPPAHGVTSAPDTRPAPGSTAPP
or
(SEQ ID No. II)
STAPPAHGVTSAPDTRPAPGSTAPP-K-palmitoyl-(G),

preferably together with an adjuvant and/or a further anti-cancer agent, wherein said liposomal formulation is applied not later than 180 days, preferably not later than 140 days, and most preferably not later than 98 days after completion of said chemo-radiotherapy.

Furthermore, the invention is related to a method of extending the survival time of a patient suffering from non-small cell lung cancer (NSCLC), preferably unresectable stage III NSCLC treated with a liposomal formulation comprising a lipopeptide based on the muc-1 core repeating unit selected from the group consisting of the amino acid sequences: STAPPAHGVTSAPDTRPAPGSTAPP (SEQ ID No. I) or STAPPAHGVTSAPDTRPAPGSTAPP-K-palmitoyl-(G) (SEQ ID No. II), by pre-treating the patient with concurrent or at least 10-95% timely overlapping chemo-radiotherapy which is completed at least 14-35 days, preferably 21-28 days before starting vaccination with said liposomal formulation but not later than 180 days, preferably not later than 140 days, preferably not later than 98 days, and most preferably not later than 84-98 days, wherein said extension is at least 15%, preferably at least 25%, compared to a respective treatment comprising an analogous sequential chemo-radiotherapy treatment, and at least 25%, preferably at least 35% compared to an analogous concurrent chemo-radiotherapy treatment, wherein a placebo is applied instead of the liposomal formulation, wherein radiotherapy is carried out by applying at least 40 Gy, preferably 50-120 Gy, more preferably 50-75 Gy of total radiation during chemo-radiotherapy, and chemotherapy is carried out by administering at least one platinum-based chemotherapeutic agent , selected from the group consisting of cisplatin and carboplatin together with an adjuvant, preferably MPL or Lipid A, and optionally an immune modulating agent, preferably cyclophosphamide, and/or a further anti-cancer agent, by at least two cycles, preferably 2-8 cycles, wherein one cycle is between 21 and 35 days, preferably between 21 and 28 days, and wherein the platinum-based chemotherapeutic agent is administered in daily, weekly or 2-5 weekly dose.

Finally the invention is related to the use of L-BLP25 (Stimuvax®) for the treatment of a patient suffering from unresectable stage III non-small cell lung cancer (NSCLC) by means of a combination therapy including chemo-radiotherapy followed by vaccination of the patient with L-BLP25, wherein the initial chemo-radiotherapy is concurrent or at least 10-95%, preferably 50-95% timely overlapping related to the duration of the chemotherapy, and the vaccination starts after completion of said chemo-radiotherapy not later than 98 days, preferably not later than 84 days, and wherein the chemotherapy is based on platinum-based chemotherapeutic agents, preferably cisplatin and carboplatin.

### DETAILED DESCRIPTION OF THE INVENTION

The mucin/muc-1 peptide according to the invention is the mature human glycoprotein directed to the muc-1 antigen and comprises the muc-1 core repeating peptide unit of the following 20 amino acids:

STAPPAHGVTSAPDTRPAPG
TAPPAHGVTSAPDTRPAPGS
APPAHGVTSAPDTRPAPGST
PPAHGVTSAPDTRPAPGSTA
PAHGVTSAPDTRPAPGSTAP
AHGVTSAPDTRPAPGSTAPP
HGVTSAPDTRPAPGSTAPPA
GVTSAPDTRPAPGSTAPPAH
VTSAPDTRPAPGSTAPPAHG
TSAPDTRPAPGSTAPPAHGV
SAPDTRPAPGSTAPPAHGVT
APDTRPAPGSTAPPAHGVTS
PDTRPAPGSTAPPAHGVTSA
DTRPAPGSTAPPAHGVTSAP
TRPAPGSTAPPAHGVTSAPD
RPAPGSTAPPAHGVTSAPDT
PAPGSTAPPAHGVTSAPDTR
APGSTAPPAHGVTSAPDTRP
PGSTAPPAHGVTSAPDTRPA
GSTAPPAHGVTSAPDTRPAP

including (i) all biologically active isoforms, variants, mutants and truncated forms thereof including glycosylated, non-glycosylated, partially glycosylated forms, and including forms with modified glycosylation and/or amino acid residue pattern; (ii) any biologically active recombinant or synthetic 20mer peptide consisting of any of the core repeating peptide units as specified above, including peptides with modified amino acid residue pattern; (iii) any biologically active recombinant or synthetic, optionally modified peptide or polypeptide based on one or more of any of the core repeating peptide units as specified above, or any biologically active recombinant or synthetic, optionally modified peptide or polypeptide comprising at least one of said core repeating peptide units and partial sequence tracks of a further repeating unit, including the 25mer peptide having the peptide sequence:

(SEQ ID No. I)
STAPPAHGVTSAPDTRPAPGSTAPP

(iv) all lipid forms of (i) to (iii), including STAPPAHGVTSAPDTRPAPGSTAPP-K-palmitoyl-(G), designated as designated as “BLP-25” and including all forms, variants, and derivatives thereof, as described in WO 1998/50527 and WO 2005/112546,

(v) all proteins, fusion proteins included, comprising the peptide or polypeptide forms as specified above,

(vi) all formulations of human muc-1, or peptide or polypeptide forms thereof as specified above, preferably any liposomal formulation, and

(vii) all formulations of muc-1 nucleic acids encoding the mature muc-1 protein, and the peptide, polypeptide and lipopetide forms as specified above in combination or association preferably via a liposome with an adjuvant, preferably MPL(3-Odesacyl-4′-monophosphoryl lipid), Lipid A, or low-toxic variants of LPS.

“L-BLP25” according to the invention is the combination or mixture of lipopetide BLP-25 or any other peptide sequence as specified above and an adjuvant, preferably MPL or Lipid A, both partners integrated in a liposomal preparation, wherein BLP-25 (or a similar lipopeptide) and the adjuvant are present in a ratio 1:1 up to 5:1 by weight, preferably approximately 2:1. The BLP-25 lipopeptide provides the antigenic specificity for the T-cell response, while the adjuvant (MPL, Lipid A) enhances the cellular immune responses. The liposomal delivery system is designed to facilitate uptake of the vaccine by antigen-presenting cells (APCs) delivering the lipopeptide into the intracellular space, finally leading to presentation of peptides vial HLA-1 and HLA-II molecules of the HLA complex. This is expected to elicit a muc-1 specific cellular immune response mediated by T-cells, including a CTL response.

The invention comprises “chemo-radiotherapy”. Chemo-radiotherapy according to the invention includes “chemotherapy”. Chemo-radiotherapy also includes “radiotherapy” carried out by radiation according to standard methods or by administration of radio-labelled compounds. According to the invention radiation is preferred.

Chemo-radiotherapy according to the invention usually starts with chemotherapy followed by radiotherapy. However, starting therapy with radiotherapy is also applicable. Chemotherapy is carried out by administration of at least one “chemotherapeutic agent”, preferably a platinum-based drug, such as cisplatin or carboplatin. According to the invention the platinum-based chemotherapeutic agents are administered daily, weekly or every 2 to 5 weeks, dependent on the dose duration and number of administrations.

Chemotherapy according to the invention comprises administration of chemotherapeutic agents which are according to the understanding of this invention a member of the class of cytotoxic agents, and include chemical agents that exert anti-neoplastic effects, i.e., prevent the development, maturation, or spread of neoplastic cells, directly on the tumor cell, and not indirectly through mechanisms such as biological response modification.

Preferred chemotherapeutic agents according to the invention which are administered in the chemo-radiotherapy settings of the invention are platinum-based agents, such as cisplatin or carboplatin. However, other chemotherapeutic agents as specified below, may be also used.

In addition further chemotherapeutic agents or other anti-cancer agents can be administered to improve efficacy of the claimed therapy. There are large numbers of anti-neoplastic agents available in commercial use, in clinical evaluation and in pre-clinical development, which could be included in the present invention for treatment of tumors/neoplasia by combination therapy. It should be pointed out that the chemotherapeutic agents can be administered optionally together with above-said antibody drug. Examples of chemotherapeutic or agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cabazitaxel, cisplatin, dacarbazine (DTIC), dactinomycin, docetaxel, mechlorethamine, streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, ketokonazole, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

In a preferred embodiment of the invention a liposomal formulation is provided, wherein the platinum-based chemotherapeutic agent is selected from the group consisting of cisplatin or carboplatin, and the non-platinum based chemotherapeutic agent is selected from the group consisting of vinorelbine, etoposide, paclitaxel, docetaxel, vindesine, gemcitabine, ifosfamide and pemetrexed.

Instead of chemotherapeutic agents, administration of immunotherapeutic agents are favorable according to the invention in addition to said platinum-based chemotherapeutic agents. Suitable immunotherapeutic agents according to the invention are, for example, anti-cancer antibodies, such as anti-VEGF(R) antibodies or anti EGFR antibodies.

In more detail, a platinum-based chemotherapeutic agent, like cisplatin and carboplatin can be combined according to the invention with drugs such as: taxanes, like pacitaxel and docetaxel; anti-angiogenic molecules such as bevacizumab, anti-metabolic agents such pemetrexed and gemcitabine; topo-isomerase inhibitors such as etoposide or irinotecan, vinca alkaloids such as vinorelbine and vinblastine, EGFR targeting agents such as cetuximab, panitumumab, erlotinib, gefitinib and afatinib, and alkylating agents such as ifosfamide.

The term “cytotoxic agent” as used herein refers to a substance that inhibits or prevents the function of cells by causing destruction of cells. The term is intended to include radioactive isotopes, chemotherapeutic agents, immunotherapeutic agents, and toxins such as enzymatically active toxins of bacterial, fungal, plant or animal origin, or fragments thereof. The term may include also members of the cytokine family, preferably I FNγ as well as anti-neoplastic agents having also cytotoxic activity.

The term “anti-cancer agent” describes all agents which are effective in cancer therapy. The term includes, cytotoxic agents, chemotherapeutic agents, and immunotherapeutic agents.

The term “concurrent or concomitant chemo-radiotherapy” means according to the invention a combination of chemotherapy and radiotherapy which are timely at least overlapping, preferably overlapping by at least 10%-15% calculated from the duration of the respective chemotherapy. Preferably chemo- and radiotherapy are overlapping more than 20%, 30%, 40%, 50%, 60%, 70%, 80% and 90%. A preferred overlapping range is between 10%-100%, preferably 20-100%, more preferably 70-100%, most preferably 50-100%. In the most preferred embodiment, radiotherapy is started after starting chemotherapy and is completed after completion of chemotherapy (100% overlap). The values indicated refer to a single patient. They may vary in a statistical consideration of a cohort of patients. The terms “concurrent” and “concomitant” are used synonymously in this document.

The term “sequential chemo-radiotherapy” means according to the invention a combination of chemotherapy and radiotherapy which are timely not overlapping at all or are overlapping by less than 10%, more preferably less than 5%, most preferably less than 1% calculated from the duration of the respective chemotherapy. In the sequential chemo-radiotherapy setting according to the invention, which is not overlapping at all, radiotherapy treatment starts preferably 1-28 days, more preferably 1-21, most preferably 7-14 days after completion of radiotherapy. The values indicated refer to a single patient. They may vary in a statistical consideration of a cohort of patients.

According to the invention the chemo-radiotherapy is applied and completed before the vaccination with said liposomal formulation is started.

Chemotherapy is applied according to the invention by at least two cycles, preferably 2-8 cycles, more preferably 2-5 cycles. One cycle is between 21 and 35 days, preferably between 21-28 days. The dose regimen of the chemotherapeutic agent, preferably the platinum-based agents is dependent on various possible patient- and drug-related conditions and properties. Usually, cisplatin is applied in doses varying from 50-120 mg/m² and per cycle. Carboplatin may be applied according to the invention in doses of 500-1500 mg per single dose and per cycle.

Radiotherapy is carried out according to the invention—as mentioned—by standard radiation, wherein a total of 40-120 Gy are applied, preferably at least 50 Gy, more preferably between 50 and 75 Gy. The radiation therapy is usually fractionated, wherein 1.5-3.5 Gy are applied per day for at least four days, preferably 5-7 days in sequence. The total radiation dose is to be applied according to the invention within 21-35 days, preferably within 28 days. If necessary or favourable, boost doses of 3.5-15 Gy, preferably 5-10 Gy can be applied at the beginning of radiation or in an intermediate interval.

According to the invention vaccination is applied after completion of the chemo-radiotherapy. The liposomal formulation comprising the lipopeptide of the invention is applied 7-35, preferably 14-28 days after completion of said radiotherapy. It could be shown that the efficacy of the vaccination treatment after chemo-radiotherapy is not influenced negatively if vaccination is not started later than 84-98 days.

Vaccination is applied according to the invention during the initial phase every 5^{th}-9^{th}, preferably every 7^{th} day. The initial phase is completed after 6-8 weeks after start. Thereafter, every 5-7 weeks, preferably every 6 weeks a further vaccination dose is applied according to the invention. One single dose of the liposomal formulation should contain according to the invention 500-1.200 μg of said lipopeptide, preferably 700-900 μg.

The chemo-radiotherapy vaccination treatment can be accompanied by administration of an agents that is capable to modulate the immune system. By, for example, applying a relatively low dose of cyclophosphamide between 100-400 mg/m²preferably 250 mg/m² the immune system of the patient can be activated or enhanced. Usually, a single dose before start of the vaccination, as a rule 1 to 5 days, preferably 2-5 days, should be sufficient to be effective.

### SHORT DESCRIPTION OF THE FIGURES

FIG. 1: Primary endpoint Overall Survival/all populations (mITT)
Placebo versus L-BLP25
mITT: modified intention-to-treat population

FIG. 2: Overall Survival: subgroup analyses 2

FIG. 3: Overall survival in concurrent chemo-radiotherapy (mITT)

FIG. 4: Overall survival in sequential chemo-radiotherapy (mITT)

FIG. 5: Overall survival by concurrent vs sequential chemo-radiotherapy (mITT)

FIG. 6: Overall survival: Subgroup analyses, mITT, concurrent vs sequential chemo-radiotherapy 1

FIG: 7: Overall survival: Subgroup analyses, mITT, concurrent vs sequential chemo-radiotherapy 2

FIG. 8: Primary and secondary endpoints concurrent vs sequential chemo-radiotherapy

FIG. 9: Timing of chemotherapy and radiotherapy relative to first diagnosis,

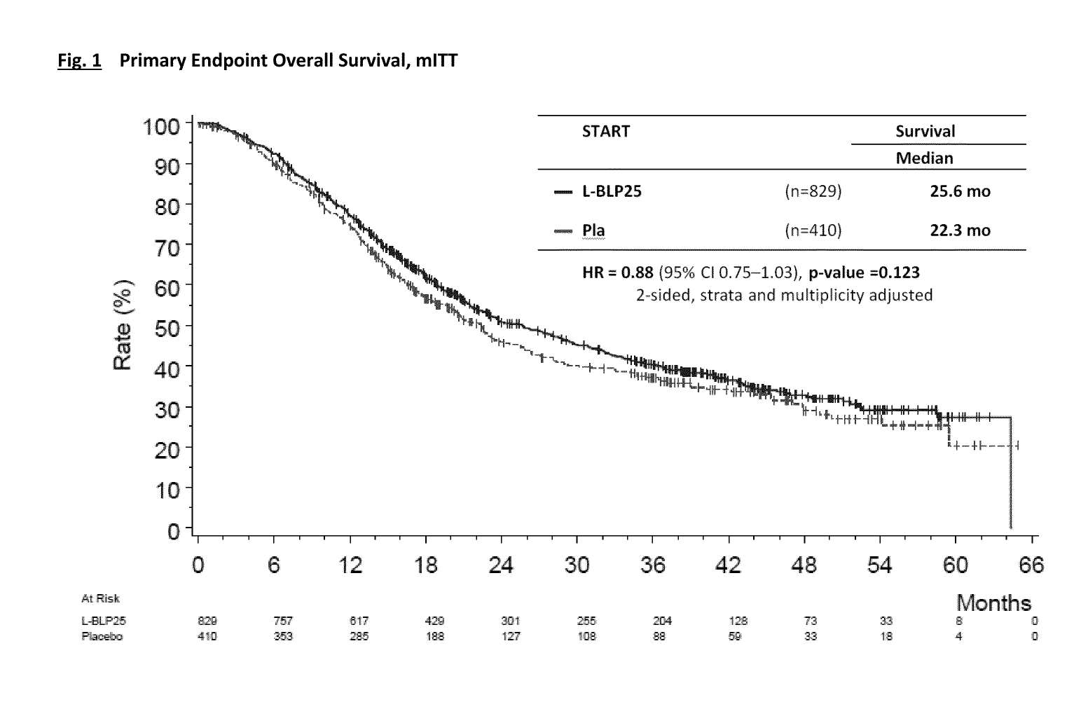

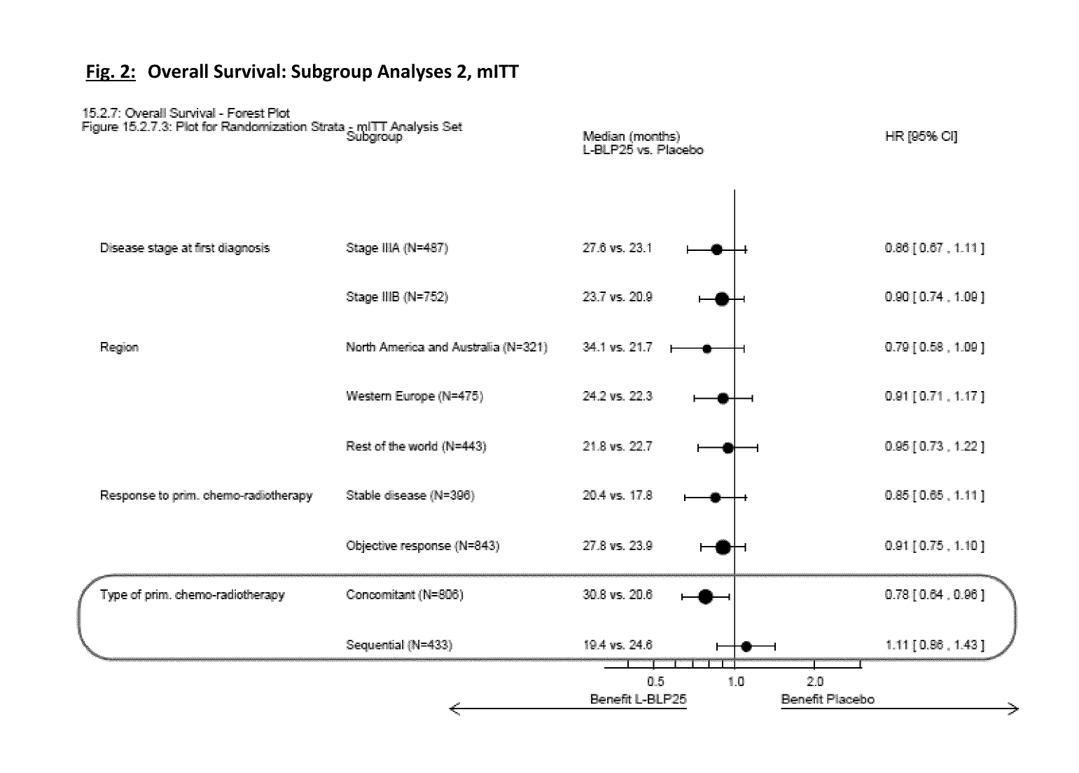

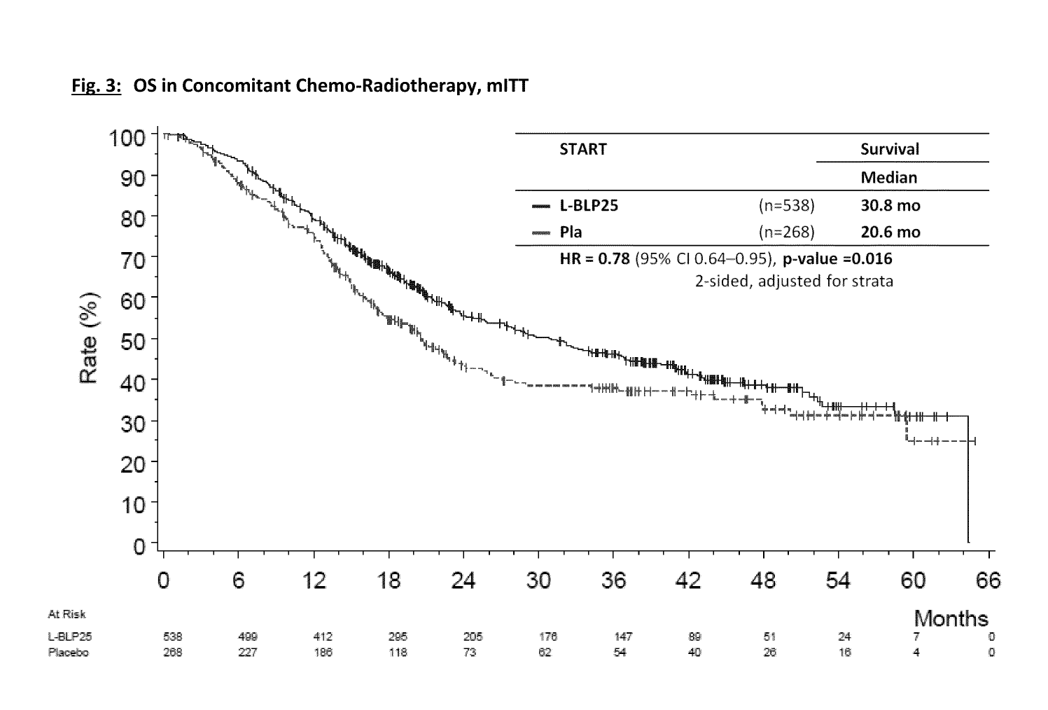

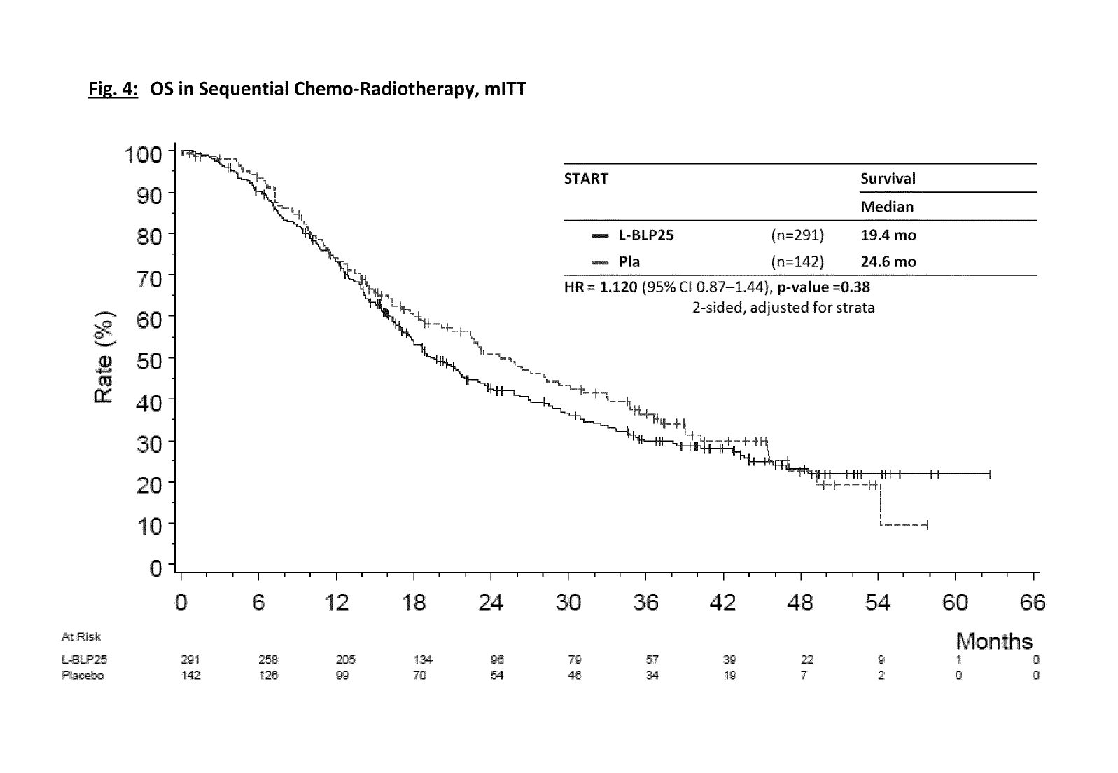

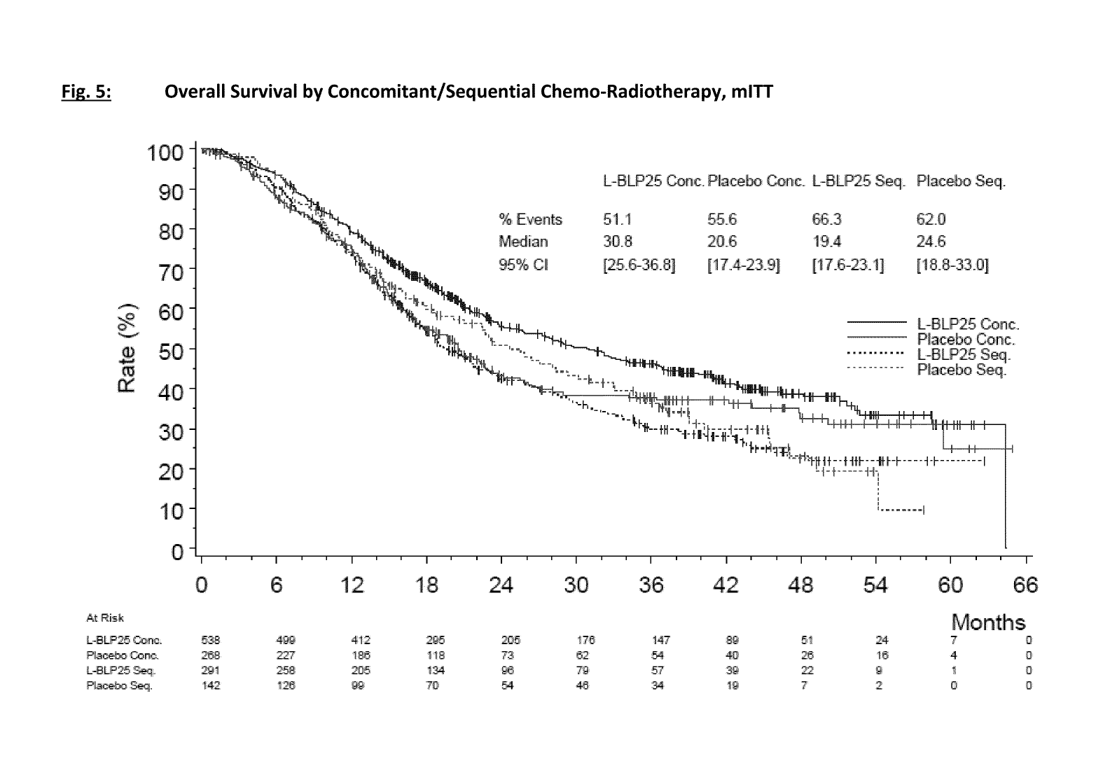

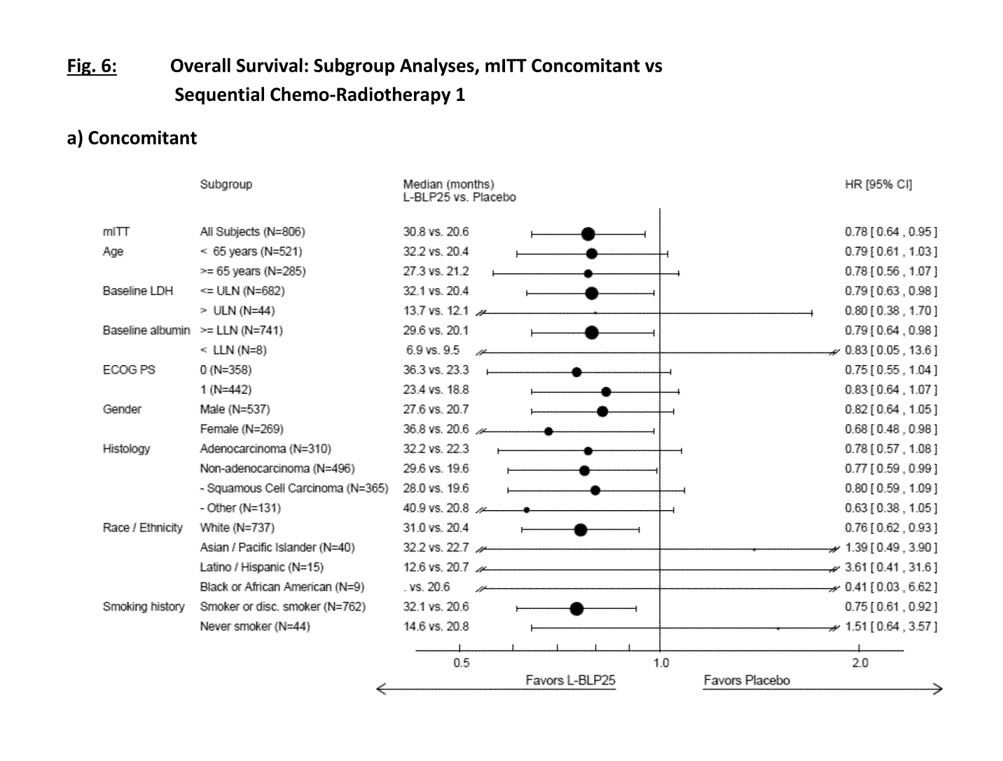

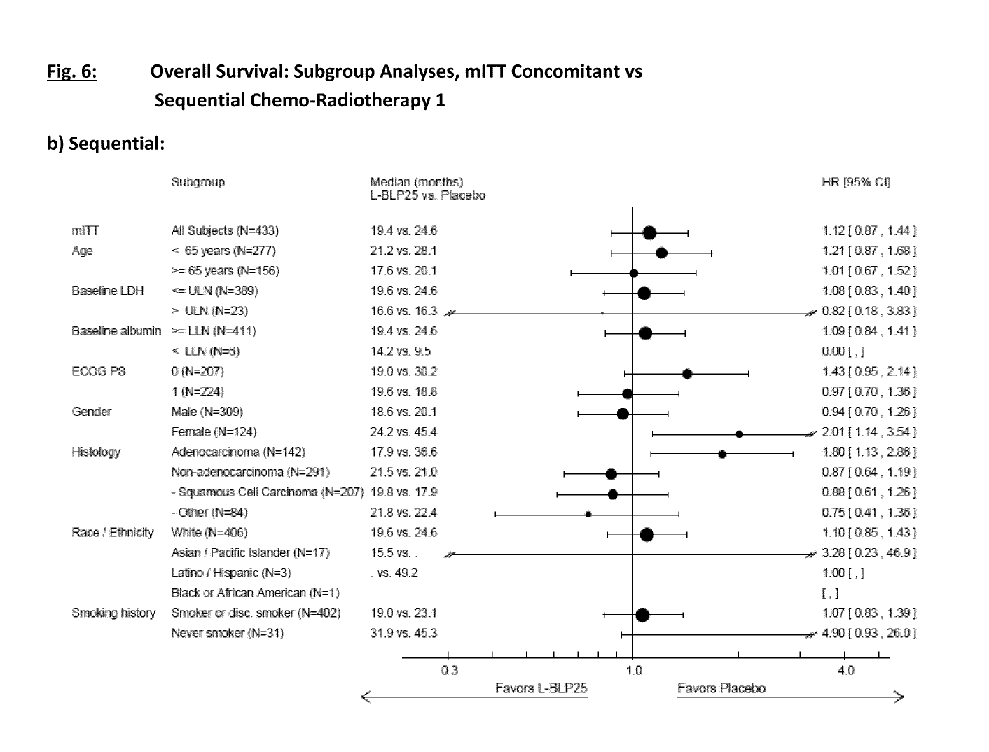

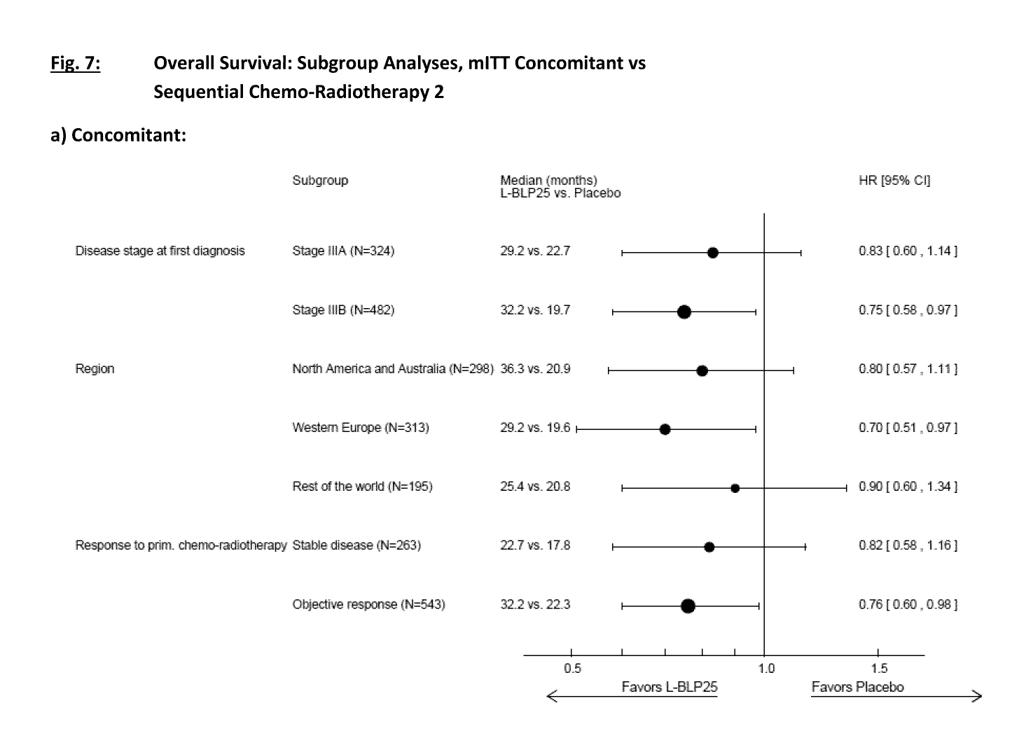

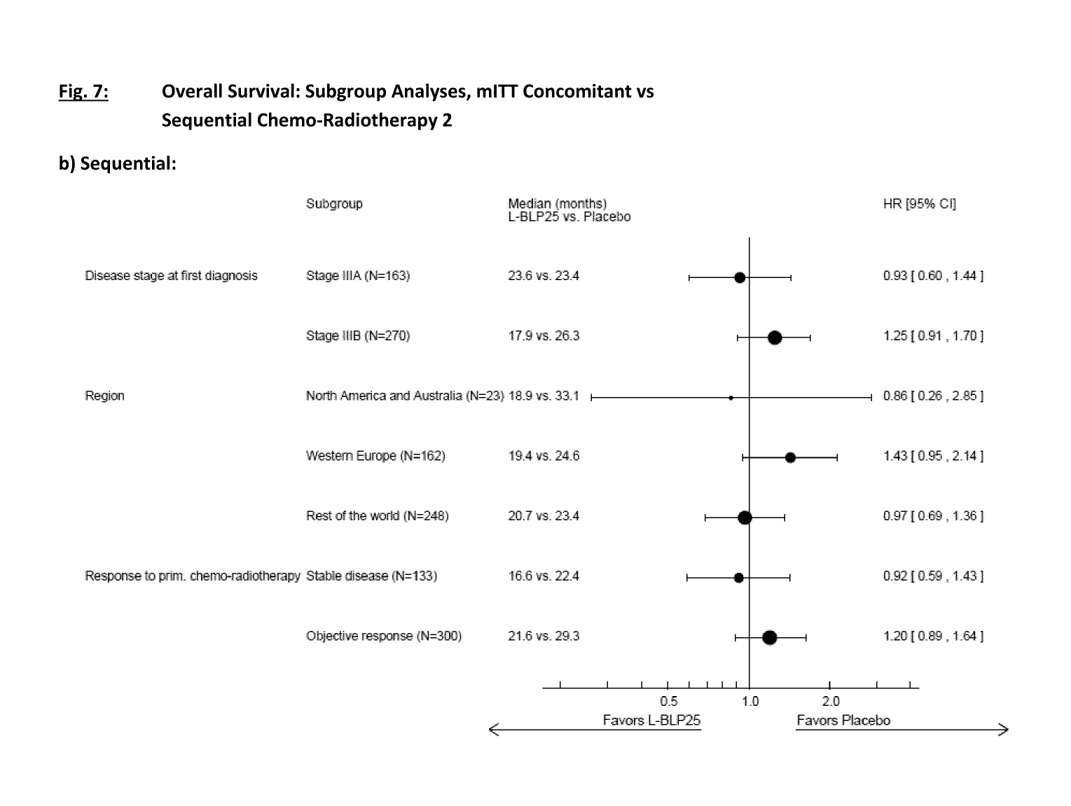

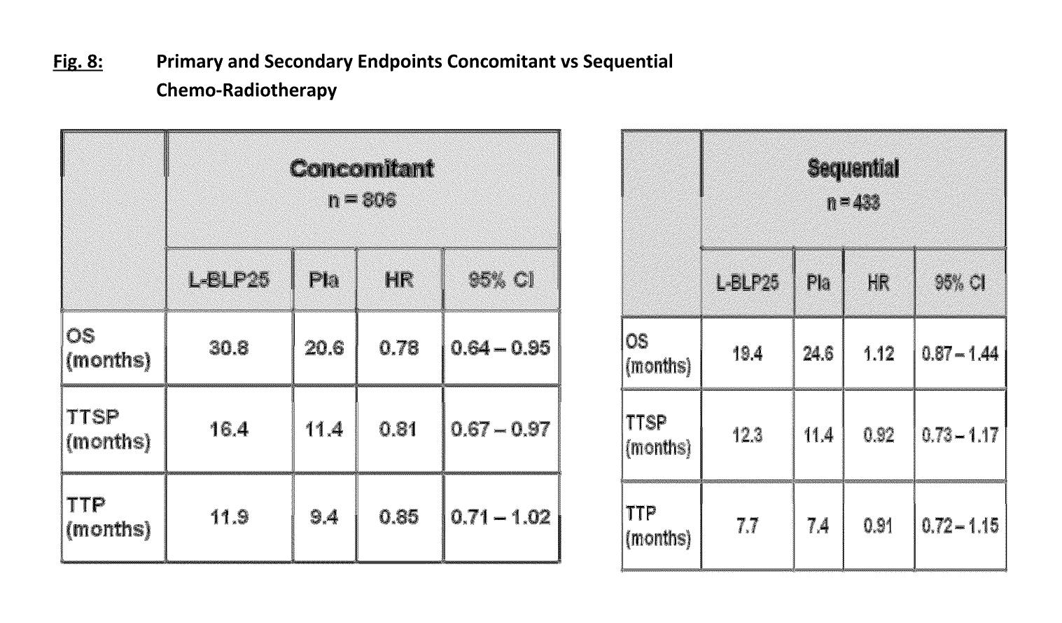

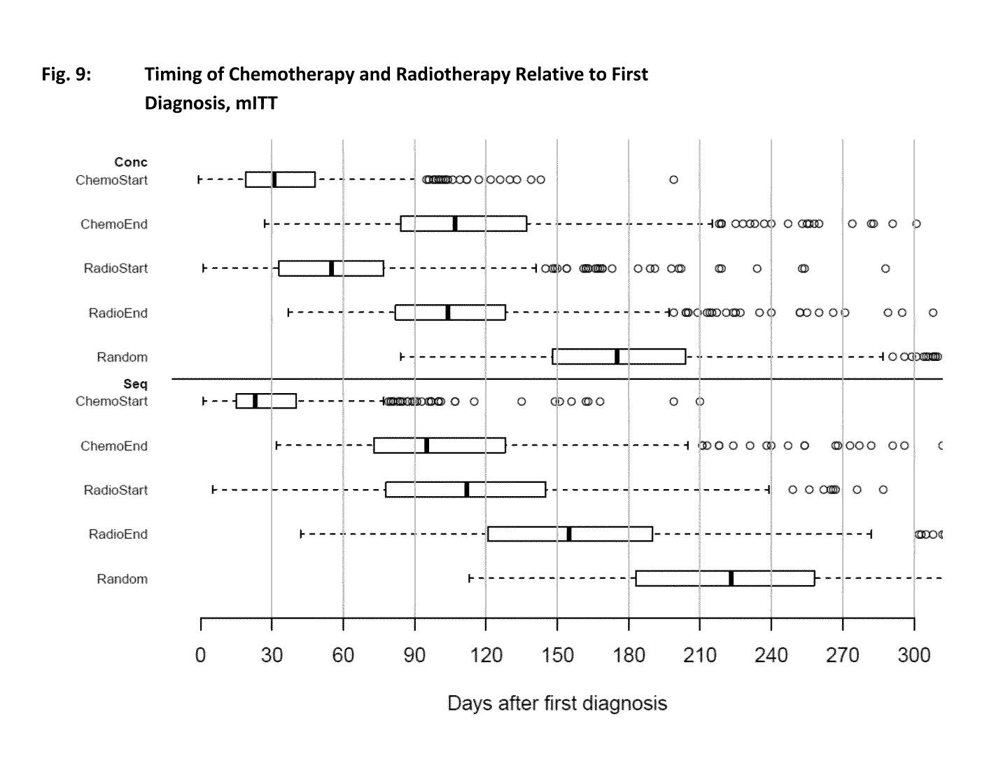

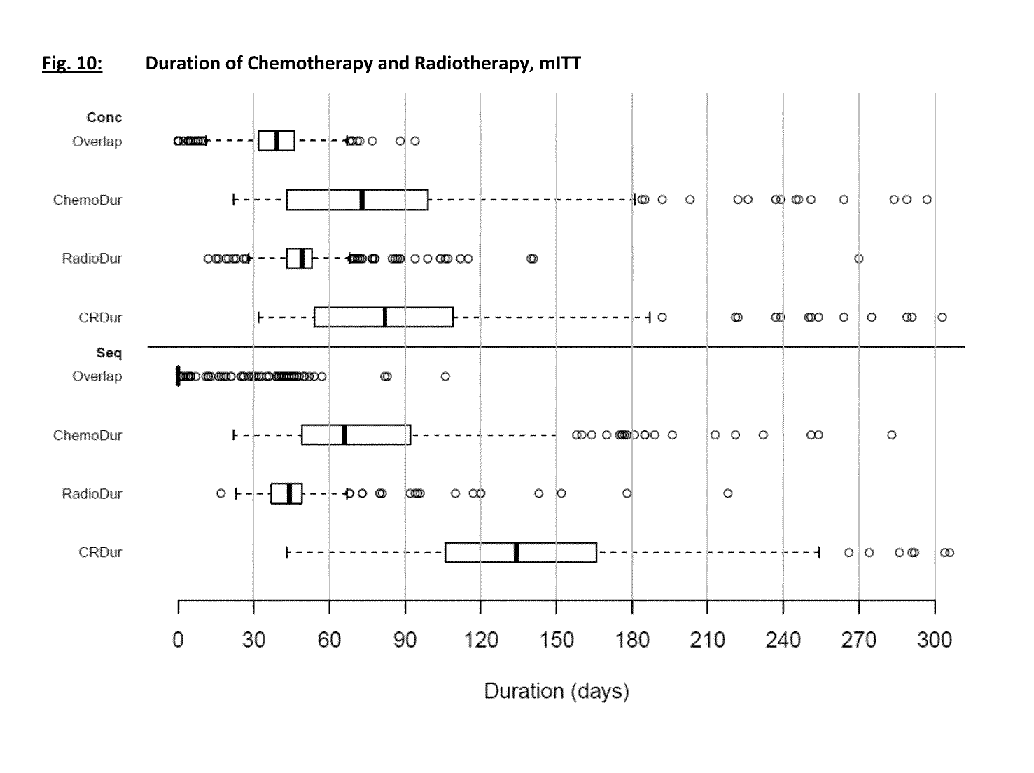

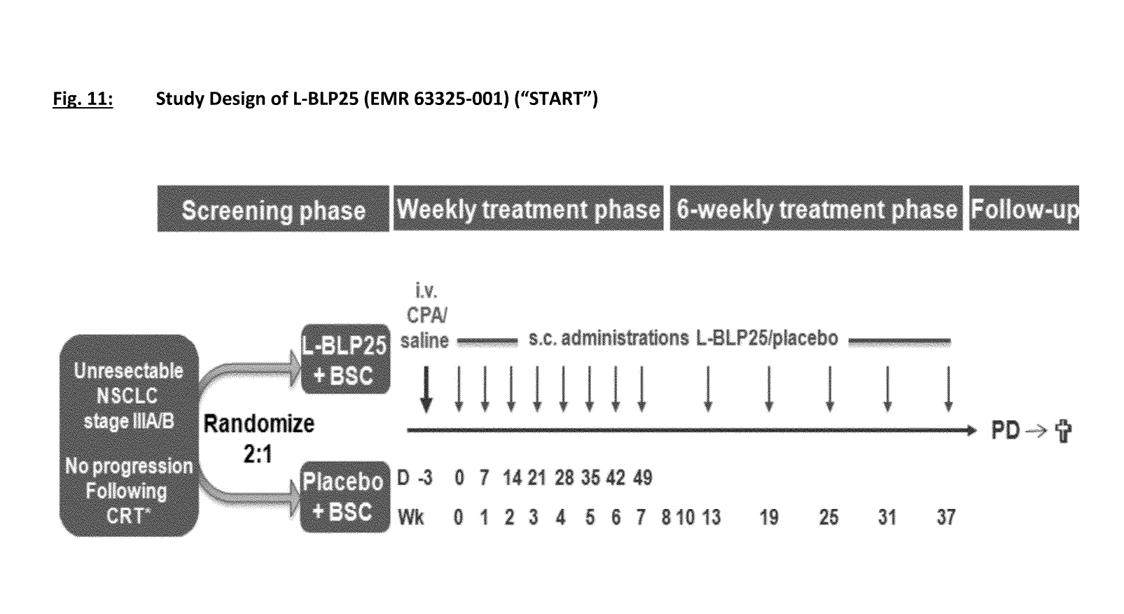

Conc Concurrent chemo-radiotheray

Seq Sequential chemo-radiotheray

ChemoStart Start day of chemotherapy relative to first diagnosis

ChemoEnd Stop day of chemotherapy relative to first diagnosis

RadioStart Start day of radiotherapy relative to first diagnosis

Radio End Stop day of radiotherapy relative to first diagnosis

Random Randomization relative to first diagnosis

The box-plots show the start and stop of chemo- and radiotherapy relative to the date of first diagnosis by randomization strata concurrent versus sequential chemoradiotherapy. The boxes stretch from the lower to the upper quartile and the mark in the box symbolizes the median, hence the box represents the mid 50% of the data.

It can be seen that concurrent and sequential chemo-radiotherapy differ overall with regard to the start of the radiotherapy. In the concurrent group radiotherapy starts on average shortly after start and before end of chemotherapy and ends approximately at the same time as the chemotherapy. In the sequential group radiotherapy starts on average shortly after completion of chemotherapy, i.e. chemotherapy and radiotherapy and administered sequentially.

The time from end of radiotherapy until randomization is comparable for the concurrent and sequential groups. Selected descriptive statistics are presented below:

| | | |
|---|---|---|
| Statistics | Concurrent | Sequential: |

Start day of chemotherapy relative to first diagnosis

| | | |
|---|---|---|
| N | 799 | 426 |
| Mean ± SD | 38.93 ± 69.05 | 32.92 ± 29.29 |
| Median (Q1-Q3) | 31 (20-48) | 23.5 (15-40) |

| | | |
|---|---|---|
| Statistics | Concurrent | Sequential |

Start day of radiotherapy relative to first diagnosis

| | | |
|---|---|---|
| N | 800 | 426 |
| Mean ± SD | 63.96 ± 76.81 | 116.25 ± 57.10 |
| Median (Q1-Q3) | 55 (33-77) | 112 (78-145) |

FIG. 10: Duration of chemotherapy and radiotherapy (mITT)

Conc Concurrent chemo-radiotheray

Seq Sequential chemo-radiotheray

Overlap Overlap of platinum chemotherapy and radiotherapy (days)

ChemoDur Duration of chemotherapy (days)

RadioDur Duration of radiotherapy (days)

CRDur Duration of chemo-radiotherapy (days)

It can be seen that the duration of the chemotherapy and radiotherapy components are comparable between concurrent and sequential groups.

The overall duration of the entire chemo-radiotherapy differs substantially between concurrent and sequential chemo-radiotherapy due to the concurrent or sequential administration of the two treatment components. This is reflected in the plot in the overlapping of platinum chemotherapy and radiotherapy—the box-plot for the overlap in the sequential group is all zero, i.e. all values from minimum, Q1, median and up to Q3 are 0 meaning no overlap. On the other hand the box-plot for the overlap in the concurrent group is indicating substantial concurrent administration with a median overlap of 39 days (Q1 32 days, Q3 46 days. Further descriptive statistics are given below:

| | | |
|---|---|---|
| Statistics | Concurrent | Sequential |

Chemotherapy duration (days)

| | | |
|---|---|---|
| N | 803 | 429 |
| Mean ± SD | 78.65 ± 43.38 | 75.50 ± 42.35 |
| Median (Q1-Q3) | 73 (43-99) | 66 (49-92) |

| | | |
|---|---|---|
| Statistics | Concurrent | Sequential |

Radiotherapy duration (days)

| | | |
|---|---|---|
| N | 804 | 429 |
| Mean ± SD | 49.62 ± 14.05 | 45.29 ± 18.04 |
| Median (Q1-Q3) | 49 (43-53) | 44 (37-49) |

Chemoradiotherapy duration (days)

| | | |
|---|---|---|
| N | 801 | 428 |
| Mean ± SD | 88.26 ± 42.25 | 140.19 ± 49.17 |
| Median (Q1-Q3) | 82 (54-109) | 134 (106-166) |

Overlap of platinum chemotherapy and radiotherapy (days)

| | | |
|---|---|---|
| N | 806 | 432 |
| Mean ± SD | 37.40 ± 13.90 | 6.04 ± 15.37 |
| Median (Q1-Q3) | 39 (32-46) | 0 (0-0) |

N = Subjects with available dates,
SD = standard deviation,
Q1 = lower quartile,
Q3 = upper quartile

FIG. 11: Study design of L-BLP25 (EMR 63325-001) (“START”)

Primary endpoint: Overall survival

Key secondary endpoints:
1. Time to symptom progression (TTSP) as measured by the Lung Cancer Symptom Scale (LCSS)
2. Time to progression (TTP) as determined by the investigator
3. Safety
*≧2 cycles of platinum-based chemotherapy; radiation ≧50 Gy

### EXAMPLE

L-BLP25 is a MUC1 antigen specific cancer immunotherapy. Here, the results report results from the phase III START study of L-BLP25 in patients (pts) not progressing after primary chemoradiotherapy (CRT) for stage III NSCLC.

This following summarizes the key results of the 100% events analysis of the START trial. All analyses are based on a dataset with clinical cut-off date of 8 Aug. 2012.

Methods and Endpoints

The design and objectives of this trial are described in the clinical trial protocol and in the statistical analysis plan (SAP) V 2.0. In brief, subjects with unresectable stage III NSCLC who have demonstrated either stable disease or objective response following primary chemo-radiotherapy (concomitant or sequential) were randomized 2:1 either to cyclophosphamide and L-BLP25 (investigational group) or to placebo (control group), respectively, in a double-blinded fashion. The randomization was stratified by disease stage (stage IIIA or IIIB), response to primary chemo-radiotherapy (stable disease or objective response), type of primary chemo-radiotherapy (concomitant or sequential), and region (1: North America [Canada, US] and Australia, 2: Western Europe, or 3: ROW [Mexico, Central and South America, Eastern Europe and Asia]). The purpose to select these stratification factors was related to prognostic factors in stage III NSCLC). Subjects in both treatment groups in addition received best supportive care according to the investigator's discretion. The primary variable of this trial was survival duration. The trial was powered with 90% to detect a significant HR of 0.77 at significance level alpha 0.05 (2-sided) assuming a median survival of 20 months in the control group.

The protocol was amended to modify the primary analysis population, which is in principle based on the intention-to-treat (ITT) population (n=1513) but under prospective exclusion of all subjects randomized during the 6 months (=26 weeks) period prior to the clinical hold (n=274). These subjects were excluded regardless of the actual survival outcome (modified ITT or mITT population). The rationale for this change was the assumption that an uninterrupted initial treatment with L-BLP25 of at least 6 months would produce a clinically relevant effect. The modified ITT (mITT) as primary analysis population and the SAP V2.0 was agreed upon with the FDA under a Special Protocol Assessment agreement, and was considered to be acceptable by the MEB, MHRA, MPA and the PEI (HAs of the Netherlands, UK, Sweden and Germany, respectively) in the context of Scientific Advice procedures.

Subject disposition:

Between initiation of screening in January 2007 and end of recruitment on 15 Nov. 2011, 1908 subjects were screened and 1513 were randomized (ITT population) to the L-BLP25 active treatment group (n=1006, 66.5%) or to the placebo treatment group (n=507, 33.5%). The safety population consists of a total of 1501 subjects with 1024 subjects in the L-BLP25 group and 477 subjects in the placebo group. The difference of 12 subjects between the ITT and the safety population reflects subjects who had been randomized but who had not started treatment. Of note, 24 subjects in the safety analysis set who had been randomized to the placebo group but received at least one administration of cyclophosphamide or L-BLP25 (major protocol violation) were evaluated in the active treatment group. Also, the placebo group of the safety analysis set contains 1 subject originally randomized to the L-BLP25 treatment group who received a saline pre-infusion only.

From January 2007 to November 2011, 1513 pts with unresectable stage III NSCLC that did not progress after CRT (platinum based chemo and ≧50 Gy) were randomized (2:1; double-blind) to L-BLP25 (806 μg lipopeptide) or placebo (PBO) SC weekly×8 then Q6 weeks until disease progression or withdrawal. Cyclophosphamide 300 mg/m²×1 or saline was given 3 days prior to first L-BLP25/PBO dose. Primary endpoint was overall survival (OS).

The primary analysis population (n=1239) was defined prospectively to try to account for a clinical hold by excluding pts randomized 6 months (m) before the hold. Arms were balanced for baseline characteristics. Median age was 61 y; 38.2% had stage IIIA and 61.3% IIIB; 65% had concurrent and 35% sequential CRT. Median OS was 25.6 m with L-BLP25 vs. 22.3 m with PBO (adjusted HR 0.88, 95% CI 0.75-1.03, p=0.123). Secondary endpoints time-to-progression and time-to-symptom-progression support consistency of results: HR 0.87 (95% CI 0.75-1.00, p=0.053) and 0.85 (95% CI 0.73-0.98, p=0.023). In predefined subgroup analyses, pts with concurrent CRT (n=806) had median OS of 30.8 m (L-BLP25) vs. 20.6 m (PBO; HR 0.78, 95% CI 0.64-0.95, p=0.016), while median OS with sequential CRT was 19.4 m (L-BLP25) vs. 24.6 m (PBO; HR 1.12, 95% CI 0.87-1.44, p=0.38; interaction p=0.032, Cox PH model). Sensitivity analyses revealed that there was no OS benefit in pts randomized 6 m before the hold (HR 1.09, CI 0.75-1.56, p=0.663). L-BLP25 was well tolerated with no safety concerns identified and no emergent evidence of immune related adverse events.

L-BLP25 maintenance therapy in stage III NSCLC was well tolerated, but did not significantly prolong OS. Sensitivity analyses showed a smaller treatment effect due to the clinical hold, suggesting that longer uninterrupted treatment with L-BLP25 is required. Clinically meaningful prolongation of OS was observed in the predefined subgroup of pts with primary concurrent CRT.

Out of the 1024 subjects treated in the L-BLP25 group 6 subjects discontinued treatment after the initial cyclophosphamide infusion and 1018 subjects were treated further. In the placebo group 6 out of 477 subjects discontinued treatment after the initial saline infusion and 471 subjects were treated with placebo. Median duration of treatment was 32.4 weeks in the

L-BLP25 group and 26.6 weeks in the placebo group (safety analysis set). Median number of vaccinations administered was 11 both in the L-BLP25 group and in the placebo group (safety analysis set).

The primary objective of this trial, i.e. to demonstrate a statistically significant prolongation of overall survival with L-BLP25 treatment assuming a true HR of 0.77 in the population under study, was not met.

Primary endpoint results—mITT population,

| | L-BLP25 (N = 829) | | Placebo (N = 410) | |
|---|---|---|---|---|
| Events, n (%) | 468 | (56.5) | 237 | (57.8) |
| Median survival time (months, 95% CI) | 25.6 | (22.5, 29.2) | 22.3 | (19.6, 25.5) |
| HR (95% CI), p-value, stratified model, multiplicity | 0.88 (0.75, 1.03), 0.123 | | | |
| adjusted | | | | |
| HR (95% CI), p-value, stratified model, | 0.89 (0.76, 1.04), 0.1566 | | | |
| unadjusted for multiplicity | | | | |
| 1 year survival rate in % (95% CI), subjects at risk | 77.0 (74.0, | 79.8), 617 | 74.7, (70.1, | 78.7), 285 |
| 2 year survival rate in % (95% CI), subjects at risk | 50.8 (47.1, | 54.4), 301 | 45.9 (40.6, | 51.1), 127 |
| 3 year survival rate in % (95% CI), subjects at risk | 40.2 (36.4, | 43.9), 204 | 37.0 (31.7, | 42.3), 88 |
| Censoring reasons, n (%) | | | | |
| Censored at cutoff date (administrative) | 310 | (37.4) | 133 | (32.4) |
| Last date known alive before cutoff | 4 | (0.5) | 2 | (0.5) |
| Lost to follow-up | 12 | (1.4) | 8 | (2.0) |
| Withdrawal of consent | 35 | (4.2) | 30 | (7.3) |

Median follow-up time: 39.9 months in the L-BLP25 group, 37.7 months in the placebo group

Overall survival results in randomization strata and subgroups:

The Forest plot in the Figures shows overall survival results for predefined baseline characteristics and randomization strata, respectively, in the mITT population. These baseline characteristics and randomization strata were defined a priori because of the known or assumed prognostic impact on survival time of NSCLC patients. For each of the illustrated baseline characteristics and randomization factors the HR estimate including 95% CI is displayed (for the randomization strata an unstratified Cox model with treatment as single factor was used). The HR estimate is depicted by a filled circle and the size of the circle is proportional to the subgroup sample size.

In nearly all subgroups a favorable effect of L-BLP25 over placebo (HR<1) was observed except for tumor histology adenocarcinoma, and sequential chemo-radiotherapy). In small subgroups like Asian/Pacific Islander and Latino/Hispanic or Never Smokers a treatment effect in favor of placebo was seen in addition (HR>1) but these groups are too small for a meaningful interpretation. The most prominent subgroups consist of the prospectively defined randomization stratum differentiating prior concomitant chemo-radiotherapy and sequential chemo-radiotherapy. The concomitantly pretreated subgroup with 806 out of 1239 subjects (65%) showed a positive effect from L-BLP25 treatment (mOS 30.8 vs. 20.6 months, adjusted HR 0.78, [95% CI 0.64-0.95], p=0.016), whereas in the sequentially pretreated subgroup this favorable effect of L-BLP25 was not observed and placebo seemed to be more favorable although the confidence interval of the HR covered unity (mOS 19.4 vs. 24.6, adjusted HR 1.12, [95% CI 0.87-1.44]).

Overall survival (OS) estimates in the randomization strata of subjects with prior concomitant and with prior sequential chemoradiotherapy

| Concomitant prior chemoradiotherapy | L-BLP25 (N = 538) | | Placebo (N = 268) | |
|---|---|---|---|---|
| Events, n (%) | 275 | (51.1) | 149 | (55.6) |
| Median survival time (months, 95% CI) | 30.8 | (25.6, 36.8) | 20.6 | (17.4, 23.9) |
| HR (95% CI), p-value, stratified model | 0.78 (0.64, 0.95), 0.0156 | | | |
| 1 year survival rate in % (95% CI), subjects | 79.1 (75.3, | 82.3), 412 | 75.1 (69.3, | 79.9), 186 |
| at risk | | | | |
| 2 year survival rate in % (95% CI), subjects | 55.5 (50.9, | 59.9), 205 | 43.3 (36.6, | 49.7), 73 |
| at risk | | | | |
| 3 year survival rate in % (95% CI), subjects | 46.2 (41.3, | 50.9), 147 | 37.8 (31.2, | 44.4), 54 |
| at risk | | | | |

| Sequential prior chemoradiotherapy | L-BLP25 (N = 291) | | Placebo (N = 142) | |
|---|---|---|---|---|
| Events, n (%) | 193 | (66.3) | 88 | (62.0) |
| Median survival time (months, 95% CI) | 19.4 | (27.6, 23.1) | 24.6 | (18.8, 33.0) |
| HR (95% CI), p-value, stratified model | 1.12 (0.87, 1.44), 0.3816 | | | |
| 1 year survival rate in % (95% CI), subjects | 73.3 (67.8, | 78.1), 205 | 74.1 (65.8, | 80.6), 99 |
| at risk | | | | |
| 2 year survival rate in % (95% CI), subjects | 42.4 (36.3, | 48.3), 96 | 50.8 (41.6, | 59.2), 54 |
| at risk | | | | |
| 3 year survival rate in % (95% CI), subjects | 29.7 (24.0, | 35.6), 57 | 36.3 (27.5, | 45.1), 34 |
| at risk | | | | |

Time to Progression by prior chemo-radiotherapy: Analyses of TTP were repeated in the stratum of prior chemo-radiotherapy. In the stratum of subjects with prior concomitant chemo-radiotherapy a HR of 0.85 was observed ([95% CI 0.71-1.02], p=0.078 not adjusted for multiplicity). In contrast, in subjects with prior sequential chemo-radiotherapy the treatment effect in favor of L-BLP25 was less clear (HR 0.91, [95% CI 0.72-1.15], p=0.437 not adjusted for multiplicity). These observations were in line with the observations made for overall survival.

Time to progression (TTP) estimates in the randomization strata of subjects with prior concomitant and with prior sequential chemo-radiotherapy

| Concomitant prior chemoradiotherapy | L-BLP25 (N = 538) | | Placebo (N = 268) | |
|---|---|---|---|---|
| Events, n (%) | 345 | (64.1) | 175 | (65.3) |
| Median TTP (months, 95% CI) | 11.9 | (10.0, 14.2) | 9.4 | (7.2, 12.0) |
| HR (95% CI), p-value, stratified model | 0.85 (0.71, 1.02), 0.0777 | | | |
| 1 year TTP rate in % (95% CI), subjects at risk | 49.9 (45.4, | 54.2), 232 | 43.5 (37.1, | 49.6), 97 |
| 2 year TTP rate in % (95% CI), subjects at risk | 33.3 (28.9, | 37.7), 101 | 29.0 (23.1, | 35.3), 42 |
| 3 year TTP rate in % (95% CI), subjects at risk | 27.8 (23.4, | 32.3), 70 | 23.8 (17.9, | 30.1), 31 |

| Sequential prior chemoradiotherapy | L-BLP25 (N = 291) | | Placebo (N = 142) | |
|---|---|---|---|---|
| Events, n (%) | 210 | (72.2) | 110 | (77.5) |
| Median TTP (months, 95% CI) | 7.7 | (6.6, 9.6) | 7.4 | (6.0, 10.0) |
| HR (95% CI), p-value, stratified model | 0.91 (0.72, 1.15), 0.4372 | | | |
| 1 year TTP rate in % (95% CI), subjects at risk | 38.9 (33.0, | 44.7), 97 | 38.7 (30.4, | 46.9), 47 |
| 2 year TTP rate in % (95% CI), subjects at risk | 23.5 (18.3, | 29.2), 43 | 17.0 (10.6, | 24.6), 16 |
| 3 year TTP rate in % (95% Cl), subjects at risk | 18.8 (13.8, | 24.3), 26 | 13.6 (7.9, | 21.0), 11 |

All analyses were repeated for these two prominent randomization strata of concomitant/concurrent (conc) versus sequential (seq) chemo-radiotherapy in the frame of post-hoc analyses. As shown for all predefined subgroups of sufficient size in the concomitant stratum a benefit in favor of L-BLP25 treatment was observed. Results for predefined subgroups in the sequential stratum were observed to be more heterogeneous. Of note, for subjects in the sequential subgroup who were either female or had adenocarcinoma, a HR in favor of placebo was observed in the subgroup of sequential chemo-radiotherapy. A detrimental effect for subjects in these subgroups could not be excluded and affected subjects on ongoing treatment were informed and re-consented.

Conclusions:

1513 subjects were randomized 2:1 to the L-BLP25 or placebo treatment groups, respectively, out of which 1239 subjects were considered in the primary analysis population (mITT). There were 6.0% of subjects with major protocol deviations in the mITT population.

All major baseline and disease characteristics were well balanced between the treatment groups across randomization strata. For the primary analysis of overall survival time in the mITT population 705 events were considered. Median follow-up time in the mITT population was 39.9 and 37.7 months in the L-BLP25 and placebo groups, respectively.

With a HR_{adj} of 0.88 of the stratified Cox PH model and a multiplicity-adjusted two-sided p-value of 0.123 for overall survival in favor of L-BLP25 this pivotal Phase III trial did not meet its primary objective. This multiplicity-adjusted HR translates into a 12% reduced risk for death under L-BLP25 compared to placebo. Median overall survival time was 25.6 months in the L-BLP25 group and 22.3 months in the placebo group.

Treatment was suspended in 531 subjects and 351 of them restarted treatment after the lift of this clinical hold with a mean treatment suspension of approximately 5 months (152.7 days).

Pre-specified sensitivity analyses to assess the impact of the clinical hold showed a higher HR closer to unity in the ITT and a HR>1 in the subgroup of subjects excluded from the primary analysis population (ITT-mITT) as compared to the mITT. Further post-hoc sensitivity analyses with varying exclusion windows analogue to the principle applied in the mITT indicated that the HR improved in favor of L-BLP25 with wider time windows excluding more subjects where treatment was suspended by the clinical hold. This is in line with an analysis of subjects recruited after the clinical hold, only, which also showed a more favorable treatment benefit compared to the mITT results (n=331, HR 0.83 [95% CI 0.58-1.19]). The survival results seen in these populations are in line with the assumption that continuous uninterrupted treatment with L-BLP25 during an extensive period of exposure is important for conferring a clinical treatment effect. Furthermore, these results seem to support the notion that the treatment effect of L-BLP25 in this trial may be underestimated because the modification of the primary analysis population by this exclusion may not have sufficiently compensated for the lack of treatment during the clinical hold. I.e., a worsening of the potential treatment effect may be assumed not only for the excluded population, but also of other subjects in whom treatment was interrupted during the clinical hold at a later stage of treatment with L-BLP25.

Subgroup analyses by randomization strata and other pre-defined subsets revealed a treatment benefit in favor of L-BLP25 in subjects previously treated with concomitant chemo-radiotherapy, whereas in sequentially pretreated subjects a trend for longer survival times were observed in the placebo group. In detail, in the subgroup of subjects previously treated with concomitant chemo-radiotherapy a HR of 0.78 with [95% CI 0.64-0.95] was observed (n=806 out of 1239, mOS 30.8 vs. 20.6 months, p=0.016 two-sided). In contrast, the subgroup of subjects with prior sequential chemo-radiotherapy showed a HR of 1.12 with a [95% CI 0.87-1.44], n=433 out of 1239, mOS 19.4 vs. 24.6 months, p=0.38 two-sided). Importantly, for the subgroup of concomitantly pre-treated subjects, further subgroup analyses within this stratum revealed similar effects in favor of L-BLP25 treatment. In contrast, results for predefined subgroups in the sequential stratum were observed to be more heterogeneous.

Secondary endpoints showed HRs<1.0 (mTTSP 14.2 vs. 11.4 months, respectively, HR 0.85, p=0.023 two sided, [95% CI 0.73-0.98]; TTP 10.0 vs. 8.4 months, respectively, HR 0.87, p=0.053 two sided, [95% CI 0.75-1.00] and PFS 9.6 vs 7.7 months, HR 0.87, p=0.0359, [95% CI 0.76-0.990]). Similarly to the subgroup analyses for the primary endpoint, subjects with prior concomitant chemoradiotherapy had a tendency towards a treatment effect in favor of L-BLP25 treatment in TTSP and TTP which was more pronounced than in the sequential stratum.

The safety data from this Phase III trial confirm the positive safety profile of L-BLP25. There was no relevant difference in AE frequencies between treatment groups. Of note, potentially immune-related diseases or events occurred at similar frequencies in both treatment groups. The overall frequency of SAES and AEs leading to death was higher in the placebo group. There were no differences in the frequencies of AEs leading to permanent discontinuation of trial treatment between the treatment groups.

Although not statistically significant, a treatment effect in favor of L-BLP25 in the overall primary analysis population was observed in the primary and secondary endpoints (OS, TTSP, TTP, respectively). This observed treatment effect was more pronounced in subjects within the stratum of prior concomitant chemo-radiotherapy. The observed effect in this subgroup was clinically meaningful (HR 0.78, [95% CI 0.64-0.95], mOS 30.8 vs. 20.6 months, p=0.016). Such effect was not observed in the subgroup of subjects with prior sequential chemo-radiotherapy (HR>1). Sensitivity analyses support the possibility that the observed treatment benefit for L-BLP25 in the concomitant stratum may have been underestimated due to the impact of the clinical hold of the trial in 2010.

In conclusion, the preliminary benefit-risk assessment for further clinical development in this indication remains positive.

1. A method for treating lung cancer, comprising concurrent chemo-radiotherapy followed by vaccination with a liposomal formulation comprising a lipopeptide based on the muc-1 core repeating unit of the amino acid sequence,
(SEQ ID No. I)
STAPPAHGVTSAPDTRPAPGSTAPP
or
(SEQ ID No. II)
STAPPAHGVTSAPDTRPAPGSTAPP-K-palmitoyl-(G)
wherein the treatment causes an overall-survival (OS) and/or a time-to-progress (TTP), which is prolonged by at least 15% compared to an analogous sequential chemo-radiotherapy treatment.

2. The method according to claim 1, wherein the chemotherapy comprises a platinum-based chemotherapeutic agent.

3. The method according to claim 2, wherein the chemotherapy additionally comprises administration of a non-platinum based chemotherapeutic agent.

4. The method according to claim 2, wherein the chemotherapy is applied by at least two cycles, and wherein one cycle is between 21 and 35 days, and wherein the platinum-based chemotherapeutic agent is administered daily, weekly or every 2 to 5 weeks.

5. The method according to claim 3, wherein the platinum-based chemotherapeutic agent is cisplatin or carboplatin, and the non-platinum based chemotherapeutic agent is vinorelbine, etoposide, paclitaxel, docetaxel, videsine, gemcitabine, ifosfamide or pemetrexed.

6. The method according to claim 5, wherein 50-120 mg cisplatin per m²or 500-1500mg carboplatin per m² are applied in one cycle.

7. The method according to claim 1, wherein the radiotherapy treatment overlaps with the chemotherapy treatment.

8. The method according to claim 1, wherein at least 50 Gy of total radiation is applied.

9. The method according to claim 1, wherein the radiation therapy is fractionated, and 1.5-3.5 Gy are applied per day for at least four days in sequence.

10. The method according to claim 1, wherein radiation therapy includes boost doses of 3.5-15 Gy per day.

11. The method according to claim 1, wherein the first vaccination by said liposome formulation is applied not before 14-35 days before completion of chemo-radiotherapy but not later than 84-98 days.

12. The method according to claim 11, wherein vaccination is applied at least two times every 5-9 days during the initial phase.

13. The method according to claim 1, wherein 500-1,200 μg of said lipopeptide are applied per single dose.

14. The method according to claim 13, wherein 700-900 μg of said lipopeptide are applied per single dose.

15. The method according to claim 1, wherein an immune modulating agent is applied 2-5 days before starting vaccination treatment.

16. The method according to claim 15, wherein the immune modulating agent is able to enhance the immune response.

17. The method according to claim 16, wherein the immune modulating agent is cyclophosphamide and is applied in a single dose of 100-400 mg/m².

18. The method according to claim 1, wherein the treatment causes an overall-survival (OS) and/or a time-to-progress (TTP), which is prolonged between 15-50%.

19. The method according to claim 1, wherein the treatment causes an overall-survival (OS) and/or a time-to-progress (TTP), which is prolonged by at least 25-60% compared to an analogous concurrent chemo-radiotherapy treatment, wherein a placebo is applied instead of the liposome vaccine formulation.

20. The method according to claim 1, further comprising an adjuvant.

21. The method according to claim 20, wherein the adjuvant is selected from the group consisting of MPL(3-Odesacyl-4′-monophosphoryl lipid), Lipid A, and low-toxic variants of LPS.

22. The method according to claim 21, wherein the adjuvant is MPL, which is part of the liposomal formulation.

23. The method according to claim 22, wherein the lipopetide is based on SEQ ID NO. 2 and the MPL-lipopetide liposomal formulation is L-BLP25.

24. The method according to claim 1, wherein the lung cancer to be treated is non-small cell lung cancer (NSCLC).

25. The method according to claim 24, wherein the cancer is unresectable stage III NSCLC.

26. The method according to claim 1, wherein the formulation is applied in combination with at least a further pharmaceutically effective anti-cancer agent.

27. A method of treating a patient suffering from lung cancer comprising the following steps:
(i) applying chemo-radiotherapy to said patient, wherein said chemotherapy and said radiotherapy is carried out concurrently or at least overlapping, and
(ii) vaccinating said patient after completion of said chemo-radiotherapy at least two times every 5^{th}-9^{th} days with a liposomal formulation comprising a lipopeptide based on the muc-1 core repeating unit of the amino acid sequence
(SEQ ID No. I)
STAPPAHGVTSAPDTRPAPGSTAPP
or
(SEQ ID No. II)
STAPPAHGVTSAPDTRPAPGSTAPP-K-palmitoyl-(G),
optionally together with an adjuvant and/or a further anti-cancer agent, wherein said liposomal formulation is applied not later than 98-180 days after completion of said chemo-radiotherapy.

28. The method of claim 27, where the liposomal formulation is applied not later than 84-98 days after completion of said chemo-radiotherapy.

29. The method of claim 27, wherein the liposomal formulation is applied not before 14-35 days after completion of said chemo-radiotherapy.

30. The method of claim 29, wherein the liposomal formulation is applied not before 14-35 days after completion of said chemo-radiotherapy.

31. The method of claim 27, wherein said chemotherapy comprises platinum-based chemotherapeutic agents and is applied by at least two cycles, one cycle comprising 21 until 28 days, and wherein the platinum-based chemotherapeutic agents are administered in daily, weekly or 2-4 weekly doses.

32. The method of claim 31, wherein the platin-based chemotherapeutic agent is cisplatin that is administered in a dose of 50-120 mg per m² and per cycle, or carboplatin that is administered in a dose of 500-1500 mg per m² and per cycle.

33. The method of claim 31, wherein the chemotherapy further includes administration of at least one non-platinum based chemotherapeutic agent selected from the group consisting of vinorelbine, etoposide, paclitaxel, docetaxel, videsine, gemcitabine, ifosfamide and pemetrexed, or at least one further anti-cancer agent.

34. The method of claim 31, wherein at least 50 Gy of total radiation is applied.

35. The method of claim 31, wherein the radiation therapy is fractionated, and 1.5-3.5 Gy are applied per day for at least four days in sequence, and wherein optionally one or more boost doses of 3.5-15 Gy per day are included.

36. The method of claim 31, wherein 500-1,200 μg of said lipopeptide are applied per single dose of said vaccine formulation.

37. The method of claim 31, wherein the adjuvant is MPL (3-Odesacyl-4′-monophosphoryl lipid (MPL)) or Lipid A.

38. The method of claim 37, wherein the adjuvant is MPL which is part of the liposomal preparation, the lipopetide is based on SEQ ID NO. 2, and this MPL-lipopetide liposomal formulation is designated as L-BLP25.

39. The method of claim 31, wherein an immune modulating agent is applied 2-5 days before starting vaccination treatment.

40. The method of claim 39, wherein the immune modulating agent is cyclophosphamide in a single dose of 100-400 mg/m².

41. The method of claim 31, wherein the lung cancer to be treated is non-small cell lung cancer (NSCLC).

42. The method of claim 41, wherein the lung cancer is unresectable stage III NSCLC.

43. A method of extending the survival time of a patient suffering from non-small cell lung cancer (NSCLC) treated with a liposomal formulation comprising a lipopeptide based on the muc-1 core repeating unit of the amino acid sequence
(SEQ ID No. I)
STAPPAHGVTSAPDTRPAPGSTAPP
or
(SEQ ID No. II)
STAPPAHGVTSAPDTRPAPGSTAPP-K-palmitoyl-(G),
comprising pre-treating the patient with concurrent or at least overlapping chemo-radiotherapy which is completed at least 14-35 days before starting vaccination with said liposomal formulation but not later than 84-98 days, wherein said extension is at least 15% compared to a respective treatment comprising an analogous sequential chemo-radiotherapy treatment, and at least 25% compared to an analogous concurrent chemo-radiotherapy treatment, wherein a placebo is applied instead of the liposomal formulation,
wherein radiotherapy is carried out by applying at least 50 Gy of total radiation during chemo-radiotherapy, and chemotherapy is carried out by administering at least one platinum-based chemotherapeutic agent selected from the group consisting of cisplatin and carboplatin together with an adjuvant, and optionally an immune modulating agent, and/or a further anti-cancer agent, by at least two cycles, wherein one cycle is between 21 and 35 days, and wherein the platinum-based chemotherapeutic agent is administered in daily, weekly or 2-5 weekly doses.

44. The method of claim 43, wherein cisplatin is administered in a dose of 50-120mg per m² and per cycle, or carboplatin is administered in a dose of 500-1500 mg per m² and per cycle.

45. The method of claim 43, wherein the adjuvant is MPL, which is part of the liposomal preparation and the lipopetide is based on SEQ ID NO. 2, and 500-1,200 μg of said lipopeptide are applied per single dose of said liposomal formulation.

46. The method of claim 43, wherein the lung cancer is unresectable stage III NSCLC.


SEQUENCE LISTING (22 sequences)

SEQ ID NO: 1 - protein, 25 aa, Artificial Sequence
  tandem-repeat region of MUC1
STAPPAHGVTSAPDTRPAPGSTAPP

SEQ ID NO: 2 - protein, 25 aa, Artificial Sequence
  tandem-repeat MUC1 linked to K-palmitoyl- (glycine)
STAPPAHGVTSAPDTRPAPGSTAPP

SEQ ID NO: 3 - protein, 20 aa, Artificial Sequence
  MUC1 repeating peptide building block 1
STAPPAHGVTSAPDTRPAPG

SEQ ID NO: 4 - protein, 20 aa, Artificial Sequence
  MUC1 repeating peptide building block 2
TAPPAHGVTSAPDTRPAPGS

SEQ ID NO: 5 - protein, 20 aa, Artificial Sequence
  MUC1 repeating peptide building block 3
APPAHGVTSAPDTRPAPGST

SEQ ID NO: 6 - protein, 20 aa, Artificial Sequence
  MUC1 repeating peptide building block 4
PPAHGVTSAPDTRPAPGSTA

SEQ ID NO: 7 - protein, 20 aa, Artificial Sequence
  MUC1 repeating peptide building block 5
PAHGVTSAPDTRPAPGSTAP

SEQ ID NO: 8 - protein, 20 aa, Artificial Sequence
  MUC1 repeating peptide building block 6
AHGVTSAPDTRPAPGSTAPP

SEQ ID NO: 9 - protein, 20 aa, Artificial Sequence
  MUC1 repeating peptide building block 7
HGVTSAPDTRPAPGSTAPPA

SEQ ID NO: 10 - protein, 20 aa, Artificial Sequence
  MUC1 repeating peptide building block 8
GVTSAPDTRPAPGSTAPPAH

SEQ ID NO: 11 - protein, 20 aa, Artificial Sequence
  MUC1 repeating peptide building block 9
VTSAPDTRPAPGSTAPPAHG

SEQ ID NO: 12 - protein, 20 aa, Artificial Sequence
  MUC1 repeating peptide building block 10
TSAPDTRPAPGSTAPPAHGV

SEQ ID NO: 13 - protein, 20 aa, Artificial Sequence
  MUC1 repeating peptide building block 11
SAPDTRPAPGSTAPPAHGVT

SEQ ID NO: 14 - protein, 20 aa, Artificial Sequence
  MUC1 repeating peptide building block 12
APDTRPAPGSTAPPAHGVTS

SEQ ID NO: 15 - protein, 20 aa, Artificial Sequence
  MUC1 repeating peptide building block 13
PDTRPAPGSTAPPAHGVTSA

SEQ ID NO: 16 - protein, 20 aa, Artificial Sequence
  MUC1 repeating peptide building block 14
DTRPAPGSTAPPAHGVTSAP

SEQ ID NO: 17 - protein, 20 aa, Artificial Sequence
  MUC1 repeating peptide building block 15
TRPAPGSTAPPAHGVTSAPD

SEQ ID NO: 18 - protein, 20 aa, Artificial Sequence
  MUC1 repeating peptide building block 16
RPAPGSTAPPAHGVTSAPDT

SEQ ID NO: 19 - protein, 20 aa, Artificial Sequence
  MUC1 repeating peptide building block 17
PAPGSTAPPAHGVTSAPDTR

SEQ ID NO: 20 - protein, 20 aa, Artificial Sequence
  MUC1 repeating peptide building block 18
APGSTAPPAHGVTSAPDTRP

SEQ ID NO: 21 - protein, 20 aa, Artificial Sequence
  MUC1 repeating peptide building block 19
PGSTAPPAHGVTSAPDTRPA

SEQ ID NO: 22 - protein, 20 aa, Artificial Sequence
  MUC1 repeating peptide building block 20
GSTAPPAHGVTSAPDTRPAP